(19) United States
(12) Patent Application Publication
(10) Pub. No.: US 2023/0225667 A1
(43) Pub. Date: Jul. 20, 2023

(54) METHOD AND APPARATUS FOR OBJECTIVELY DETERMINING A FRAILTY SCORE FOR A SUBJECT

(71) Applicant: NEC Corporation, Minato-ku, Tokyo (JP)

(72) Inventors: Ni Ni SOE, Singapore (SG); Charles Chi Hin CHOY, Singapore (SG)

(73) Assignee: NEC Corporaiton, Minato-ku, Tokyo (JP)

(21) Appl. No.: 18/011,407

(22) PCT Filed: May 25, 2021
(86) PCT No.: PCT/JP2021/019697

§ 371 Date: Dec. 19, 2022

(30) Foreign Application Priority Data
Jun. 22, 2020 (SG) 1020200596IP

(51) Int. Cl. A61B 5/397; A61B 5/11; G16H 20/30

(52) U.S. Cl. CPC A61B 5/397; A61B 5/112; A61B 5/1107; G16H 20/30

(57) ABSTRACT

According to a first aspect of the present invention, there is provided a method for objectively determining a frailty score for a subject, the method comprising: deriving an attribute from sensor data including at least one of EMG data and motion data of the subject and obtained from at least one limb of the subject; quantitatively processing the attribute to determine how the subject is responding to the rehabilitation or exercises program; and objectively determining the frailty score based on the quantitative processing.

### TECHNICAL FIELD

The present invention relates broadly, but not exclusively, to a method and an apparatus for objectively determining a frailty score for a subject.

### BACKGROUND ART

Frailty is a condition associated with excess functional decline, dependency, and physical disability. Frailty individuals require considerable assistance from or full dependency on their caregivers to perform activities of daily living (ADL). ). Frailty is becoming a more important entity to clinicians and patients, especially in perioperative setting and critical care, as elderly people comprise an increasing proportion of patients presenting for surgery and to intensive care units (ICUs) worldwide because of aging population. Physical functional decline is very common after major surgery. It can lead to a decrease of independence in performing ADLs.

Preoperative physical functional status also contributes to postoperative physical functional decline. Early prehabilitation is recommended and initiated with the ultimate goal of achieving better postoperative outcome, reducing postoperative complications and faster recovery. Evidence indicates that preoperative frailty score also associated with adverse operational outcome such as increased healthcare utilization, longer hospitalization and mortality.

Individual frailty score is not only related to physical disability but also dependent on multidimensional factors such as social, psychological and physical performance. Currently, therapist and nurses perform frailty assessment in the clinic using questionnaire and requiring patient’s input.

Some clinical frailty assessment subjectively use mobility tests such as time up and go or/and six minute walk test which is not suitable for knee or hip surgery patients.

Some clinical frailty assessment also use grip strength using dynamometer as a part of functional ability.

### SUMMARY OF INVENTION

### Technical Problem

Although various frailty measurement tools have been devised, these can be of limited use in the intensive care unit or surgical setting.

Some performance-based measures, such as ‘timed-up-and-go’ tests are impossible for mechanically ventilated or acutely unwell patients to perform.

Importantly, these clinical assessment tools do not measure functional performance or ability. The ability to perform ADLs depends largely on the patient’s recovery of motor function and functional abilities. Accurate assessments of functional abilities provide important information for pre/rehabilitation planning and enable realistic goal-setting by clinicians, therapists and the patients themselves.

To accurately measure patients’ functional abilities, physiological data, such as measuring muscle strength, is critical. Muscle strength is essential for all physical activities such as activities of daily living, work, sports and maintaining posture. Reduced muscle strength is frequently apparent in older participants and creates a potential risk for a decline of activities. Long bed rest after operation induces muscle mass loss, hence reduced muscle strength and decline in physical ability. EMG measurements are very important to measure muscle strength.

Quadriceps muscle strength is very important for lower limb control due to its major contribution to dynamic activities such as walking stairs, rising from a chair, and walking and etc. The quadriceps femoris muscle plays a role in knee joint stability during gait, and functional deterioration increases the stress on the affected knee. This can lead to deterioration in quality of life in patients. Therefore, it is important to focus on the function of the quadriceps femoris muscle and to address functional deterioration from an early phase.

Since frailty score is also an important indicator to monitor patients’ functional performance and surgical outcome, especially in preoperative care, it is important to understand the relationship between the frailty score and patient’s current functional performance, as well as their recovery progress in rehabilitation treatment, in order to provide suitable treatment strategies during operation and post operation.

Clinically, quadriceps muscle strength and frailty score are measured subjectively by clinicians. However, there is a lack of automatic frailty assessment systems to measure functional ability, especially during the early stages of patients’ recovery.

Therefore, there is a need that provides reliable objective quadriceps muscle strength and objective frailty score to enhance clinical assessment in preoperative care and community dwelling.

Herein disclosed are embodiments of a device and methods for providing objective frailty assessment that addresses one or more of the above problems.

Furthermore, other desirable features and characteristics will become apparent from the subsequent detailed description and the appended claims, taken in conjunction with the accompanying drawings and this background of the disclosure.

### Solution to Problem

According to a first aspect of the present invention, there is provided a method for objectively determining a frailty score for a subject, the method comprising: deriving an attribute from sensor data including at least one of EMG data and motion data of the subject and obtained from at least one limb of the subject; quantitatively processing the attribute to determine how the subject is responding to the rehabilitation or exercises program; and objectively determining the frailty score based on the quantitative processing.

In an embodiment, the method further comprising: obtaining the sensor data from at least a quadriceps of the subject, and wherein the frailty score indicates at least one of an objective quadriceps muscle strength, range of motion, velocity of movement, similarity score of an injured limb and an injured limb and asymmetry index of an injured limb and an injured limb and some clinical information.

In an embodiment, the step of quantitatively processing the attribute includes determining at least one of a range of motion of the subject and velocity of movement of the subject, an objective muscle strength, similarity score of an injured limb and an uninjured limb and asymmetry index of an injured limb and an injured limb and an uninjured limb.

In an embodiment, the method comprises receiving time taken for the subject to get up from a sitting position, wherein the step of quantitatively processing the attribute is based on received time taken.

In an embodiment, the methods comprises receiving data indicating an amount of muscle contraction of the subject during getting up from a sitting position, wherein the step of quantitatively processing the attribute is based on repeated movement to get up from the sitting position, or predetermined period of time or a predetermined number of repetition to get up from sitting position.

In an embodiment, the method comprises receiving time taken for the subject to perform at least one of quadriceps contraction task and knee extension from a sitting position or supine position.

In an embodiment, the method comprises receiving data indicating the amount of muscle contraction and level of knee extension or quadriceps contraction task of the subject, wherein the step of quantitatively processing the attribute is based on the received data indicating the amount of muscle contraction and the level of knee extension or quadriceps contraction task during a predetermined period of time or a predetermined number of repetition.

In an embodiment, step of deriving an attribute from the sensor data comprises extracting features.

In an embodiment, the step of quantitatively processing the attribute involves an exercise program.

In an embodiment, the method comprises remotely assisting the exercise or rehabilitation program of the subject.

In an embodiment, the method comprises sending the frailty score wirelessly.

### BRIEF DESCRIPTION OF DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the invention will be better understood and readily apparent to one of ordinary skill in the art from the following written description, by way of example only, and in conjunction with the drawings, in which:

FIG. 1 shows a system to objectively determine a frailty score for a subject according to an aspect of the present disclosure.

FIG. 2 shows a block diagram of a frailty assessment server for objectively determining a frailty score for a subject according to an example.

FIG. 3 shows a block diagram of a clinical assessment module for objectively determining a frailty score for a subject according to an example.

FIG. 4 shows a block diagram of a functional performance assessment module for objectively determining a frailty score for a subject according to an example.

FIG. 5 shows a flow chart for objectively determining a frailty score for a subject according to an example.

FIG. 6 shows an example of extracting categories for objectively determining a frailty score for a subject.

FIG. 7 shows a block diagram of a frailty scoring module for objectively determining a frailty score for a subject according to an example.

FIG. 8 shows an example of how sensors placed on a subject are used to transmit data using an exercise program module.

FIG. 9 shows an example of how sensors are placed on a lower limb of a subject to detect muscle contraction.

FIG. 10 shows an example of a subject moving from an initial sitting position to a midway of sit to stand position.

FIG. 11 shows an example of where sensors are placed to detect movement of a subject who is moving from an initial sitting position to a midway of sit to stand position.

FIG. 12 shows an exemplary computing device that may be used to execute the method of FIG. 5.

### DESCRIPTION OF EMBODIMENTS

### Terms Description

Subject- a subject may be any suitable type of entity, which may include a person, a patient and a user. The term subject is used herein to identify a user or patient that requires his frailty score to be assessed. A subject who is registered to the frailty assessment server will be called a registered user. A user who is not registered to the frailty assessment will be called a non-registered user. The term subject will be used to collectively refer to both registered and non-registered users.

Frailty Assessment Server — The frailty assessment server is a server that hosts software application programs for receiving inputs, processing data and objectively assessing a frailty score. The frailty assessment server communicates with any other servers (e.g., a remote assistance server) to manage requests. The frailty assessment server communicates with a remote assistance server to assess a frailty score and facilitate situations in which exercise programs need to be managed. Frailty assessment servers may use a variety of different protocols and procedures in order to manage the data and provide a frailty score.

The frailty assessment server is usually managed by a provider that may be an entity (e.g. a company or organization) which operates to process requests, manage exercise or rehabilitation programs. The server may include one or more computing devices that are used for processing frailty score requests and providing services beyond clinical type.

A frailty assessment account — a frailty assessment account is an account of a user who is registered at a frailty assessment server. In certain circumstances, the frailty assessment account is not required to use the remote assistance server. A frailty assessment account includes details (e.g., name, address, vehicle etc.) of a user.

The frailty assessment server manages frailty assessment accounts of users and the interactions between users and other external servers, along with the data that is exchanged.

### Detailed Description

Where reference is made in any one or more of the accompanying drawings to steps and/or features, which have the same reference numerals, those steps and/or features have for the purposes of this description the same function(s) or operation(s), unless the contrary intention appears.

It is to be noted that the discussions contained in the “Background” section and that above relating to prior art arrangements relate to discussions of devices which form public knowledge through their use. Such should not be interpreted as a representation by the present inventor(s) or the patent applicant that such devices in any way form part of the common general knowledge in the art.

### The System 100

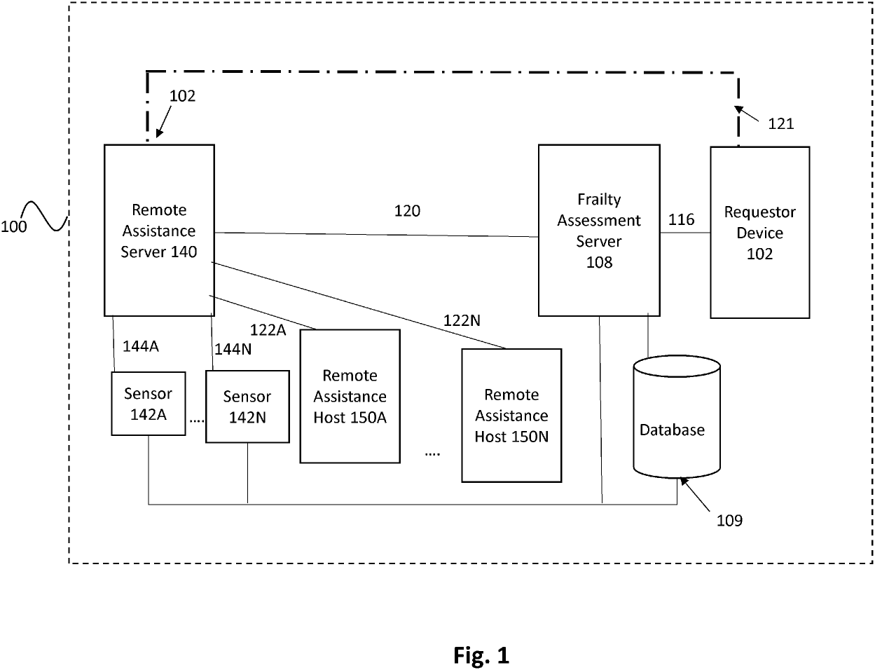

FIG. 1 illustrates a block diagram of a system 100 for objectively determining a frailty score of a subject.

The system 100 comprises a requestor device 102, a frailty assessment server 108, a remote assistance server 140, remote assistance hosts 150A to 150N, and sensors 142A to 142N.

The requestor device 102 is in communication with frailty assessment server 108 and/or a remote assistance server 140 via a connection 116 and 121, respectively. The connection 116 and 121 may be wireless (e.g., via NFC communication, Bluetooth, etc.) or over a network (e.g., the Internet). The connection 116 and 121 may also be that of a network (e.g., the Internet).

The frailty assessment server 108 is further in communication with the remote assistance server 140 via a connection 120. The connection 120 may be over a network (e.g., a local area network, a wide area network, the Internet, etc.). In one arrangement, the frailty assessment server 108 and the remote assistance server 140 are combined and the connection 120 may be an interconnected bus. It can be appreciated that the frailty assessment server 108 may be in communication with the sensors 142A to 142N. In an embodiment, the frailty assessment server 108 is that is assessable by user, via a window application on a laptop, an app on Android or iOS.

The remote assistance server 140, in turn, is in communication with the remote assistance hosts 150A to 150N via respective connections 122A to 122N. The connections 122A to 122N may be a network (e.g., the Internet).

The remote assistance hosts 150A to 150N are servers. The term host is used herein to differentiate between the remote assistance hosts 150A to 150N and the remote assistance server 140. The remote assistance hosts 150A to 150N are collectively referred to herein as the remote assistance hosts 150, while the remote assistance host 150 refers to one of the remote assistance hosts 150. The remote assistance hosts 150 may be combined with the remote assistance server 140. In an example, the remote assistance host 150 may be one managed by a hospital and the remote assistance server 140 is a central server that manages emergency calls and decides which of the remote assistance hosts 150 to forward an emergency call.

Sensors 142A to 142N are connected to the remote assistance server 140 or the frailty assessment server 108 via respective connections 144A to 144N. The sensors 142A to 142N are collectively referred to herein as the sensors 142, while the sensor 142 refers to one of the sensors 142. The connections 144A to 144N are collectively referred to herein as the connections 144, while the connection 144 refers to one of the connections 144. The connection 144 may be wireless (e.g., via NFC communication, Bluetooth, etc.) or over a network (e.g., the Internet). The sensor 144 may be one of an image capturing device, motion sensor and an EMG sensor and may be configured to send a signal, and/or receive the signal depending on the type of sensor, to at least one of the requestor device 102.

In the illustrative embodiment, each of the devices 102 and 142; and the servers 108, 140, and 150 provides an interface to enable communication with other connected devices 102 and 142 and/or servers 108, 140, and 150. Such communication is facilitated by an application programming interface (“API”). Such APIs may be part of a user interface that may include graphical user interfaces (GUIs), Web-based interfaces, programmatic interfaces such as application programming interfaces (APIs) and/or sets of remote procedure calls (RPCs) corresponding to interface elements, messaging interfaces in which the interface elements correspond to messages of a communication protocol, and/or suitable combinations thereof. For example, it is possible for the requestor device 102 to send an alert signal when a user presses a panic button on the GUI, while doing an exercise. Similarly, it is possible to make a request to obtain a frailty score using the requester device 102.

Use of the term ‘server’ herein can mean a single computing device or a plurality of interconnected computing devices which operate together to perform a particular function. That is, the server may be contained within a single hardware unit or be distributed among several or many different hardware units.

### The Remote Assistance Server 140

The remote assistance server 140 is associated with an entity (e.g. a company or organization or moderator of the service). In one arrangement, the remote assistance server 140 is owned and operated by the entity operating the server 108. In such an arrangement, the remote assistance server 140 may be implemented as a part (e.g., a computer program module, a computing device, etc.) of server 108.

The remote assistance server 140 is also configured to manage the registration of users. A registered user has a remote access account (see the discussion above) which includes details of the user. The registration step is called on-boarding. A user may use either the requestor device 102 to perform on-boarding to the remote assistance server 140.

It is not necessary to have a remote assistance account at the remote assistance server 140 to access the functionalities of the remote assistance server 140. However, there are functions that are available to a registered user. For example, it may be possible to select a suitable workout program for a user based on clinical assessment. These additional functions will be discussed below.

The on-boarding process for a user is performed by the user through one of the requestor device 102. In one arrangement, the user downloads an app (which includes the API to interact with the remote assistance server 140) to the sensor 142. In another arrangement, the user accesses a website (which includes the API to interact with the remote assistance server 140) on the requestor device 102 or the provider device 104. The user is then able to interact with the remote assistance server 140 to via the sensor that is paired with the user.

Details of the registration include, for example, name of the user, address of the user, emergency contact, blood type or other healthcare information and the sensor 142 that is authorized to update the remote assistance account, and the like.

Once on-boarded, the user would have a remote assistance account that stores all the details.

### The Requestor Device 102

The requestor device 102 is associated with a subject (or requestor) who is a party to a frailty score request that starts at the requestor device 102. The requestor may be a healthcare worker who is assisting to get data necessary to obtain a frailty assessment score. The requestor device 102 may be a computing device such as a desktop computer, an interactive voice response (IVR) system, a smartphone, a laptop computer, a personal digital assistant computer (PDA), a mobile computer, a tablet computer, and the like.

In one example arrangement, the requestor device 102 is a computing device in a watch or similar wearable and is fitted with a wireless communications interface.

### The Frailty Assessment Server 108

The frailty assessment server 108 is as described above in the terms description section.

The frailty assessment server 108 is configured to perform processes relating to objectively determining a frailty score of a subject.

### The Remote Access Hosts 150

The remote access host 150 is a server associated with an entity (e.g. a company or organization) which manages (e.g. establishes, administers) healthcare resources including exercise programs.

In one arrangement, the entity is a hospital. Therefore, each entity operates a remote access host 150 to manage the resources by that entity. In one arrangement, a remote access host 150 receives an alert signal that a subject is likely to have a low frailty score. The remote access host 150 may then arrange to send resources to the location identified by the location information included in the alert signal. For example, the host may be one that is configured to arrange for more clinical assessment.

In one arrangement, the frailty score can be automatically updated on the remote access account associated with the user. Advantageously, this allows other healthcare workers to know how the subject is doing.

### Sensor 142

The sensor 142 is associated with a user associated with the requestor device 102. More details of how the sensor may be utilised will be provided below.

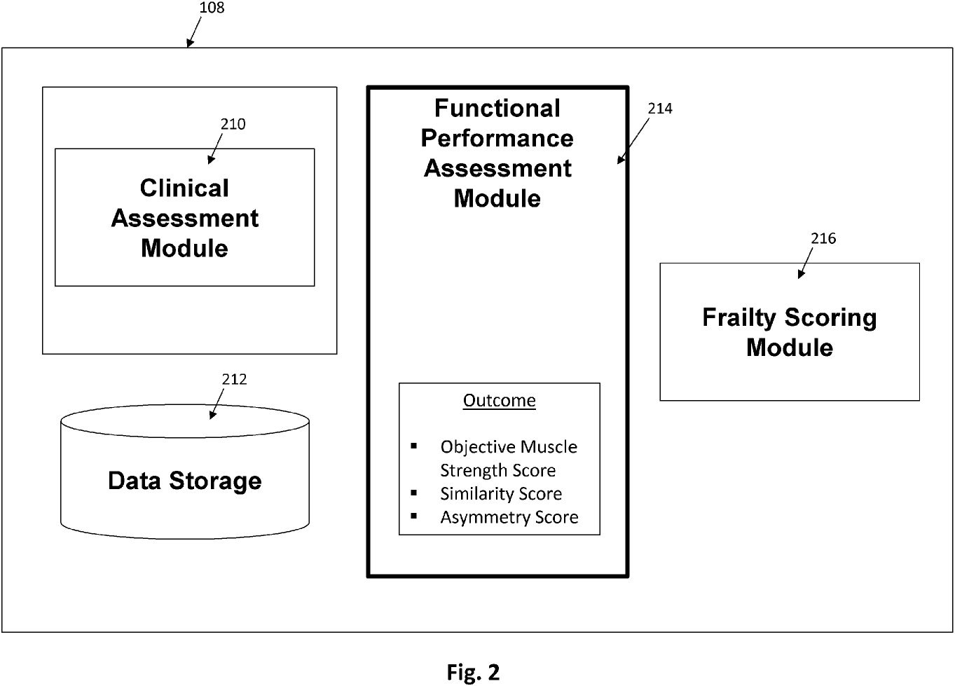

FIG. 2 shows a block diagram of a frailty assessment server 108 for objectively determining a frailty score for a subject according to an example. The frailty assessment server 108 is a server that hosts software application programs for receiving inputs, processing data and objectively assessing a frailty score. The frailty assessment server communicates with any other servers (e.g., a remote assistance server) to manage requests. The frailty assessment server communicates with a remote assistance server to assess a frailty score and facilitate situations in which exercise programs need to be managed. Frailty assessment servers may use a variety of different protocols and procedures in order to manage the data and provide a frailty score.

The frailty assessment server is usually managed by a provider that may be an entity (e.g. a company or organization) which operates to process requests, manage exercise or rehabilitation programs. The server may include one or more computing devices that are used for processing frailty score requests and providing services beyond clinical type. As shown in FIG. 2, the frailty server 108 may include a clinical assessment module 210, a data storage (or database) 212, a functional performance assessment module 214 and a frailty scoring module 216.

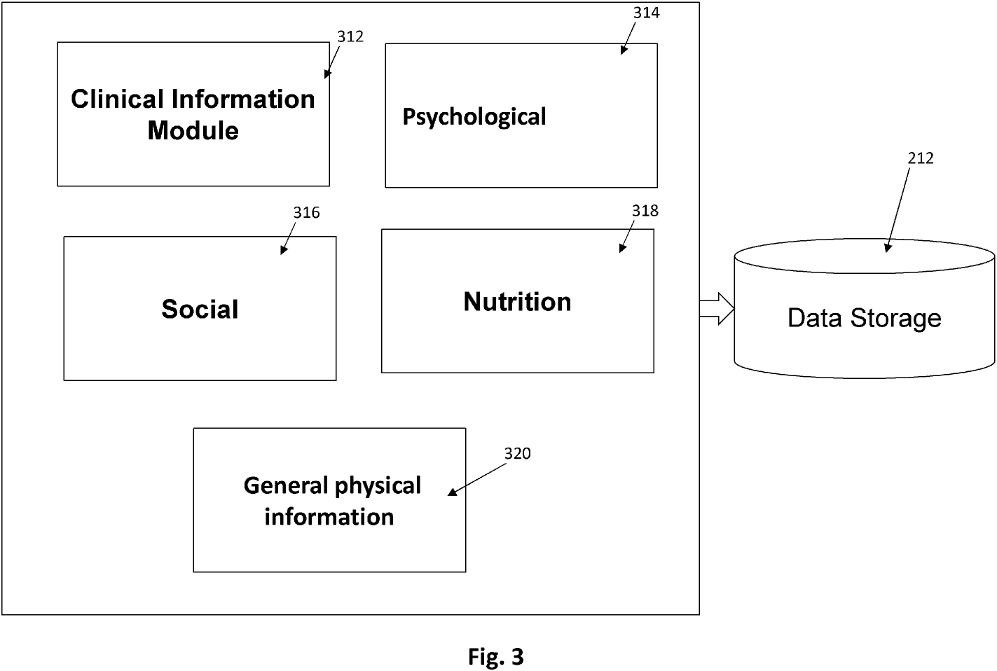

The clinical assessment module 210 is in communication with frailty performance assessment module 214 and/or a frailty scoring module 216 via a connection. The connection may be wireless (e.g., via NFC communication, Bluetooth, etc.) or over a network (e.g., the Internet). The connection may also be that of a network (e.g., the Internet). More details on each of the module will be provided in the figures below on how they work to provide an outcome that is at least one of a range of motion of the subject and velocity of movement of the subject, an objective muscle strength, similarity score of an injured limb and an uninjured limb and asymmetry index of an injured limb and an injured limb. FIG. 3 shows a block diagram of a clinical assessment module for objectively determining a frailty score for a subject according to an example.

The clinical assessment module 210 may include a clinical information module 312, a social module 316, a psychological module 314 and a nutrition module. Each of these modules is in communication with one another and a data storage 212 (as shown in FIG. 2) via a connection respectively. The connection may be wireless (e.g., via NFC communication, Bluetooth, etc.) or over a network (e.g., the Internet). The connection may also be that of a network (e.g., the Internet).

The clinical information module 312 may include information including but not limited to clinical trials, medicines, exercise programs that are being carried out and/or have been carried out.

The psychological module 314 may include information including but not limited to a psychological state of the subject. This may be linked to an account associated with the subject.

The social module 316 may include information including but not limited to those obtained on social media, new and medical journals.

The nutrition module 318 may include information including but not limited to nutritional value of food or diets in general, particularly those suitable to the subject.

The general physical module 320 may include information including but not limited to physical information of the subject. For example, the information may indicate whether or not the subject has an earlier injury. This may be linked to an account associated with the subject.

The clinical assessment module 210 includes clinical information that is necessary to provide an objective frailty score.

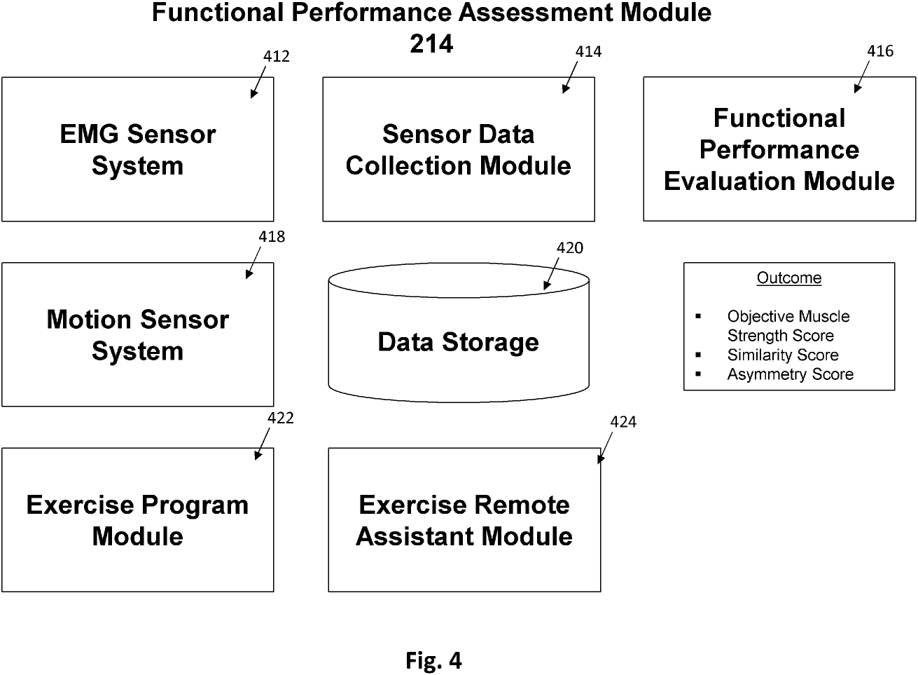

FIG. 4 a block diagram of a functional performance assessment module for objectively determining a frailty score for a subject according to an example.

The functional performance assessment module 214 may include a EMG sensor system 412, a sensor data collection module 414, a functional performance evaluation module 416, a motion sensor system 418, a data storage 420, an exercise program module 422 and an exercise remote assistance module. Each of these modules is in communication with one another via a connection respectively. The connection may be wireless (e.g., via NFC communication, Bluetooth, etc.) or over a network (e.g., the Internet). The connection may also be that of a network (e.g., the Internet).

The EMG sensor system 412 is one that receives, transmits and processes sensor data that is collected at sensor 144. The EMG sensor system 412 is used when the sensor 412 is an EMG sensor.

The motion sensor system 418 is one that receives, transmits and processes sensor data that is collected at sensor 144. The motion sensor system 418 is used when the sensor 412 is a motion sensor.

The sensor data collection module 414 is one that receives, transmits and processes sensor data that is collected at either the EMG sensor system 412 or the motion sensor system 418. The sensor data collection module 414 is used when the sensor 412 is at least one of a motion sensor and an EMG sensor.

The exercise program module 422 is one that stores a list of appropriate exercises that are suitable to determine a frailty score of the subject.

The exercise remote assistant module 424 is one that may work independently or with the exercise program module 422 to provide assistance remotely to a subject that is performing the exercise. For example, the exercise remote assistant module 424 may be one that can provide messages to the subject on how to do the exercise.

Each of these modules may be configured to communicate with a data storage 420 via a connection and each of the messages or information that passes through the modules to be stored in the data storage.

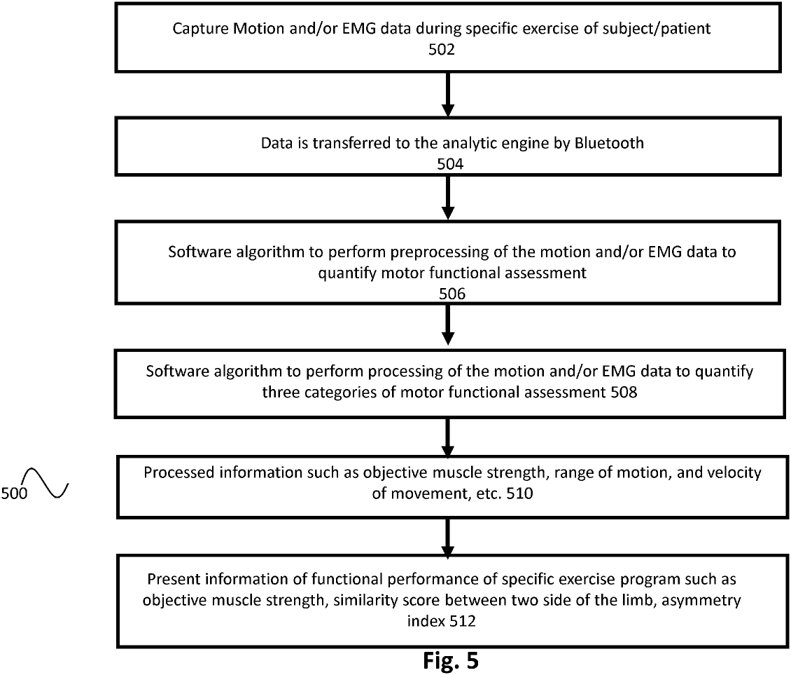

FIG. 5 shows a flow chart 500 for objectively determining a muscle strength score for a subject and functional performance of a subject according to an example. At step 502, motion and/or EMG data is captured while the subject is doing a specific exercise. The method then proceeds onto step 504.

At step 504, the data that is captured at step 502 is transmitted wirelessly for further processing. The method then proceeds onto step 506.

At step 506, software algorithm is used to perform preprocessing of the motion and/or EMG data to quantify motor functional assessment. The method then proceeds onto step 508.

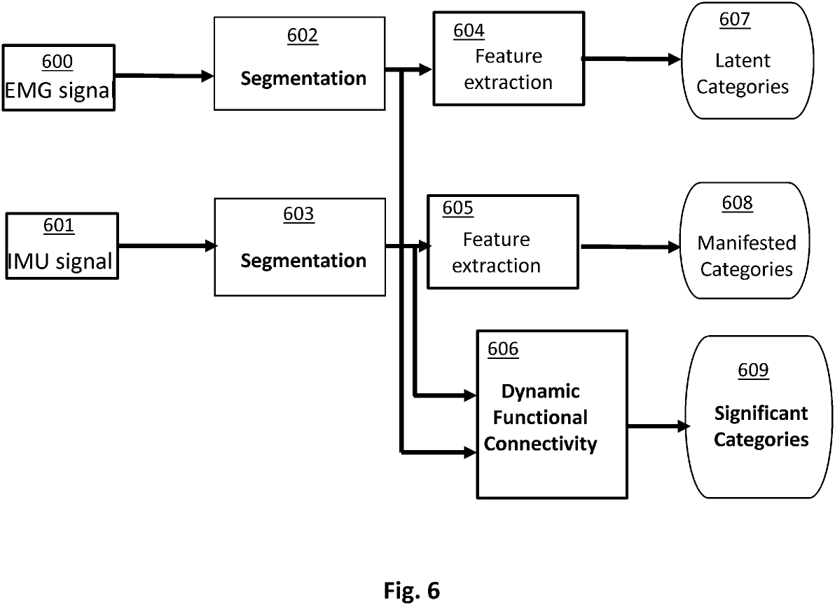

At step 508, software algorithm is used to perform processing of the motion and/or EMG data to quantify three categories of motor functional assessment. More information will be provided in below in relation to FIG. 6. The method then proceeds onto step 510.

At step 510, information relating to at least one of objective muscle strength, a range of motion, and velocity of movement will be processed. The method then proceeds onto step 512.

At step 512, information relating to at least one functional performance of specific exercise program will be provided as an output such as at least one of a range of motion of the subject and velocity of movement of the subject, an objective muscle strength, similarity score of an injured limb and an uninjured limb and asymmetry index of an injured limb and an uninjured limb.

FIG. 6 shows an example of extracting categories for objectively determining a frailty score for a subject.

At 600, an EMG signal that is received is sent to 602 for segmentation. Features are extracted at 604 and latent categories are identified. Latent categories are features derived from the EMG sensor data of a subject.

At 601, an initial measurement unit (or IMF) signal that is received is sent to 603 for segmentation. Features are extracted at 605 and manifested categories are identified. Manifested categories are features derived from the motion sensor data of a subject.

Additionally or alternatively, subsequent to segmentation 603, dynamic functional connectivity 606 will be carried out to identify significant categories at 609. The significant categories are those that are significant in computing the output of frailty score. Significant categories are the features derived from the combination of EMG and motion sensor data.

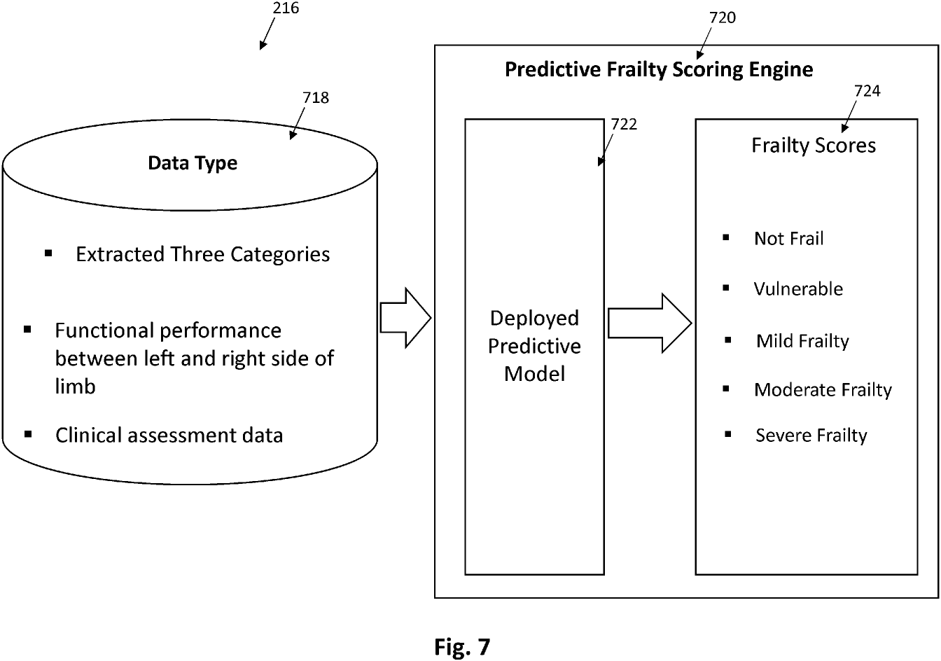

FIG. 7 shows a block diagram of a frailty scoring module for objectively determining a frailty score for a subject according to an example. The frailty scoring module 216 is part of the frailty assessment server 108, as shown in FIG. 2.

Data that has been extracted, for example, latent categories, manifested categories and significant categories, will be sent to predictive frailty scoring engine 720. Other information such as clinical data and functional performance between the left and right side of the limb will also be sent to the predictive frailty scoring engine 720.

The predictive frailty scoring engine 720 includes a deployed predictive model 722 that is configured to determine a frailty score (namely “Not frail”, “Vulnerable”, “Mild Frailty”, “Moderate Frailty” and “Severe Frailty”.

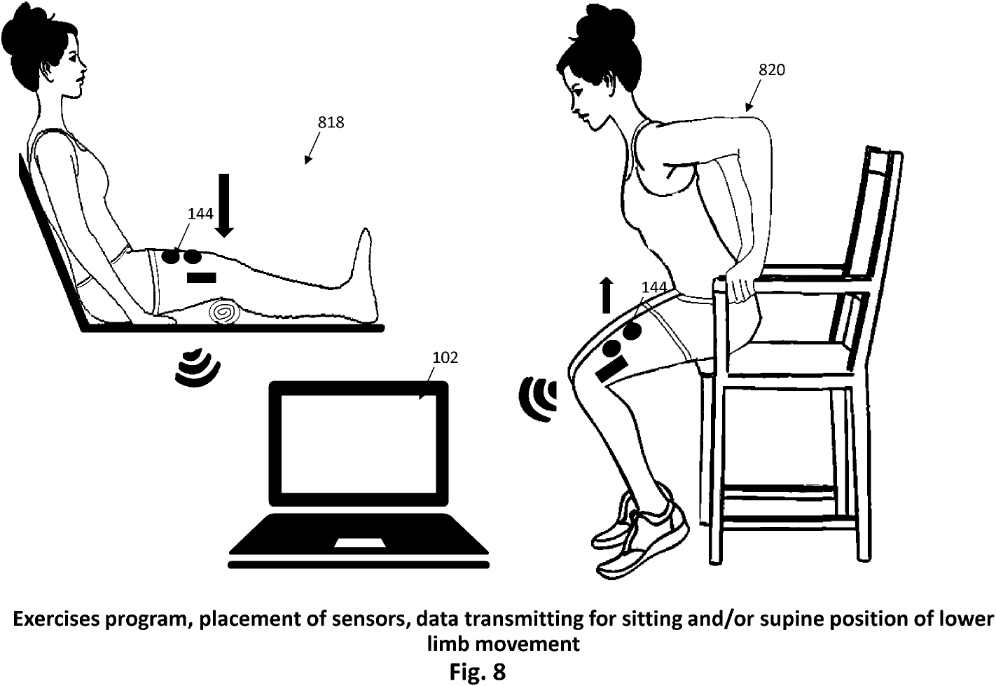

FIG. 8 shows an example of how sensors placed on a subject are used to transmit data using an exercise program module.

FIG. 8 shows an example of a device (or a requestor device 102 as shown in FIG. 1) for obtaining data of one or more muscle groups of a body for rehabilitation or exercise program according to an example embodiment. The device 102 is configured to receive data for a sensor device (or sensor 144 shown in FIG. 1) The sensor 144 includes a support member, two or more electrodes positioned on the support member and two or more location identifiers that are also positioned on the support member. The support member may be elastic such that it can be stretched to adapt to different sizes of the body of a user. Being stretchable, the support member may be adapted to circumferentially enclose a portion of the body relating to the one or more muscle groups regardless of the body size. In addition, the support member 102 may be made from a soft, non-rigid material such as polyester or vinyl. Alternatively, the sensors may be configured to attach to the subject directly.

The two or more electrodes may be configured to obtain data corresponding to the one or more muscle groups while the two or more location identifiers can be permanently fixed or removably attached to the support member. Each of the two or more electrodes may be a disposable electrode such as a “peel-and-stick” electrode typically used in electrocardiogram. Other types of electrodes, such as dry electrodes, surface electrodes, gelled electrodes, needle electrodes and/or fine wire electrodes are also possible. The two or more electrodes may obtain data from the one or more muscle groups to detect medical abnormalities, activation level of the muscle, or to analyse the biomechanics of the user.

In the embodiment where the two or more electrodes may be removable from the support member, the device 144 may include two or more electrode mounts configured to receive the two or more electrodes respectively. The function of the electrode mounts is to better secure the electrodes. In an embodiment, the two or more electrodes may obtain electrical signals from the one or more muscle groups to assist in muscle activity. In an alternate embodiment, the two or more electrodes may be configured to transmit or receive data to the one or more muscle groups of the body. For example, the two or more electrodes may transmit electrical signals to the one or more muscle groups to assist in muscle stimulation. The transmission of electrical signals for muscle stimulation may assist to relieve pain for the one or more muscle groups.

Each of the two or more location identifiers may identify a location to position one of the two or more electrodes to obtain data corresponding to the one or more muscle groups of the body. Each of the two or more location identifiers may be located at a predetermined length relative to an edge of the support member. For example, a location identifier may be at a length of approximately 5.5 centimeters. It can be appreciated that the dimensions of the location identifiers may vary depending on the muscle group of the body. For example, location identifier may be positioned at a different length from the edge of the support member for obtaining data from a muscle group of a limb.

The device 144 may also include fastening means so that the support member can be removably secured against the body. The fastening means may include but not limited to: a hook and loop fastener (e.g. a Velcro® fastener), a buckle fastener and/or an adhesive fastener. It can be appreciated that other fastening means may be applicable such that the device 144 can be secured and removed from the body with minimal effort. For example, when the subject is sitting down and foam-rolling as shown in 818, the device 144 will still be secured. In another example, when the subject is in a position of getting up from a chair as shown in 820, the device 144 will still be secured.

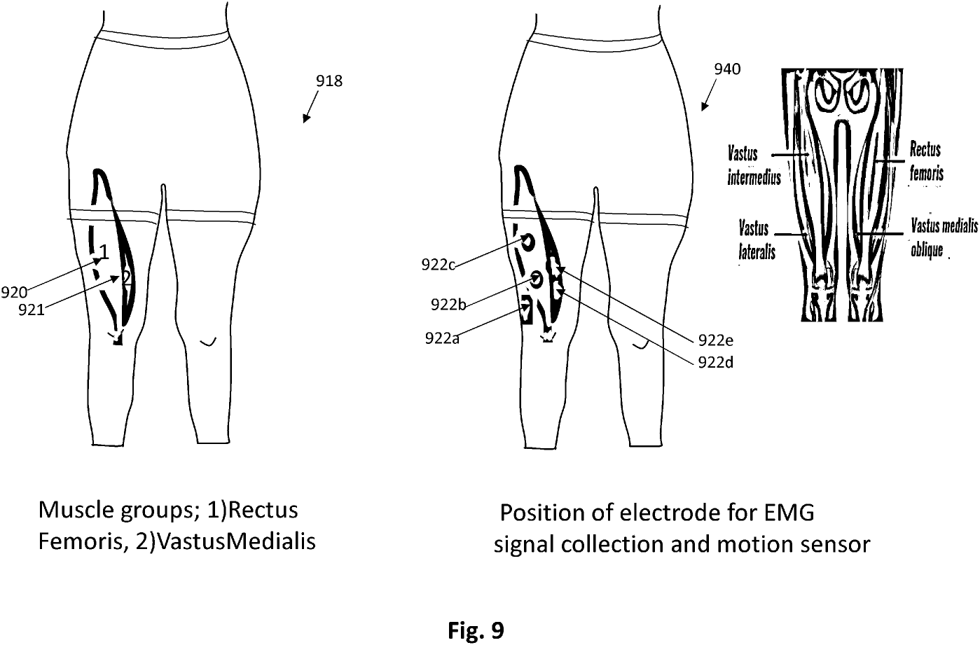

FIG. 9 illustrates muscle groups of a lower limb in which data is obtained by the two or more electrodes according to an example embodiment. In this embodiment, the one or more muscle groups of the body may refer to the muscle groups of a lower limb, such as a thigh of a subject. For example and as shown in 940, electrodes 922a, 922b, 922c, 922d, and 922e may be positioned to obtain data corresponding to the rectus femoris muscle group 920 and vastus medialis muscle group 921 that is located in the thigh of the subject. This can be shown on 918 in FIG. 9.

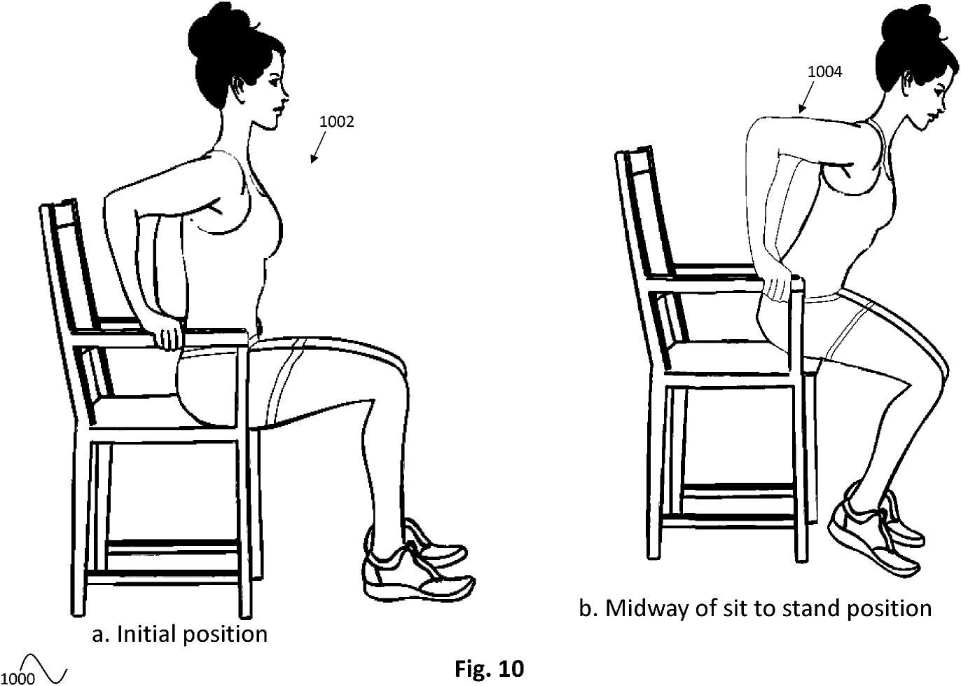

FIG. 10 shows an example of a subject moving from an initial sitting position 1002 to a midway of sit to stand position 1004. In an example, the disclosure works to obtain inputs from the muscle groups of the subject in order to objectively determine a frailty score of the subject. More information can be seen below on how that may be done.

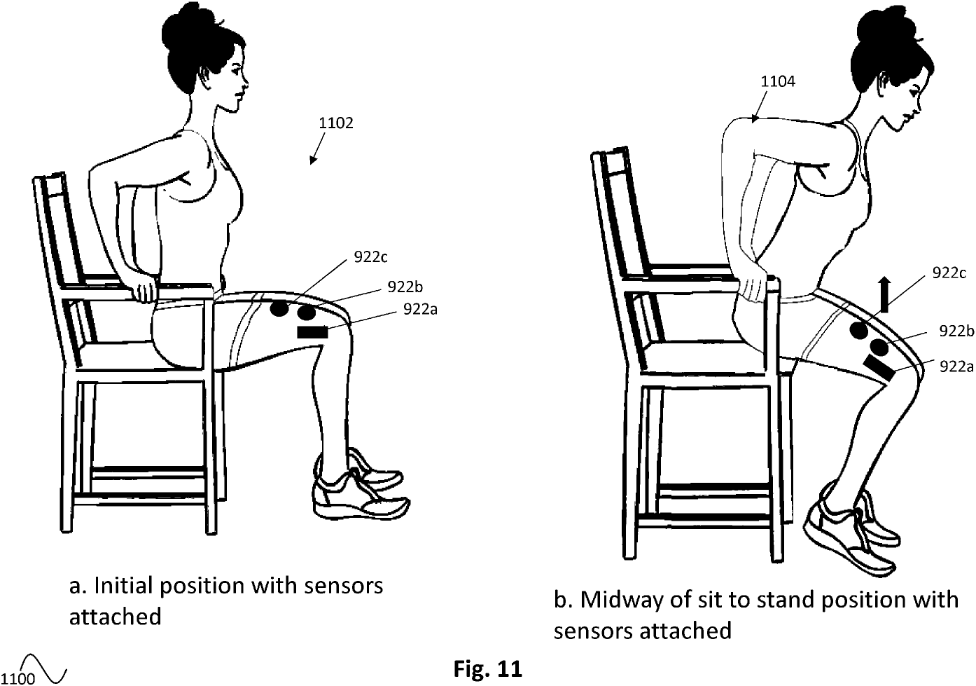

FIG. 11 shows an example of where sensors are placed to detect movement of a subject who is moving from an initial sitting position 1102 to a midway of sit to stand position 1104. In an example, the disclosure works to obtain inputs from the muscle groups of the subject in order to objectively determine a frailty score of the subject. For example, electrodes 922a, 922b and 922c, may be positioned to obtain data corresponding to the rectus femoris muscle group 920 and vastus medialis muscle group that is located in the thigh of the subject. The subject may be advised by one of the modules in the frailty score server 108 to perform an exercise program and electrical signals relating to the muscle groups may be obtained as she moves from one position to another.

In an example, the sensor is configured to obtain the sensor data from at least a quadriceps of the subject, and wherein the frailty score indicates an objective quadriceps muscle strength, range of motion, velocity of movement, similarity score of an injured limb and an injured limb and asymmetry index of an injured limb and an injured limb and some clinical information

Additionally, the step of quantitatively processing the attribute includes determining at least one of a range of motion of the subject and velocity of movement of the subject, an objective muscle strength score of measured muscle group of a subject and similarity score of an injured limb and an uninjured limb.

In an example, time taken for the subject to get up from a sitting position is obtained, wherein the step of quantitatively processing the attribute is based on received time taken.

In an example, receiving data indicating the amount of muscle contraction and level of knee extension or quadriceps contraction task of the subject is obtained, wherein the step of quantitatively processing the attribute is based on the received data indicating the amount of muscle contraction and the level of knee extension or quadriceps contraction task during a predetermined period of time or a predetermined number of repetition.

In an example, data indicating a level of knee extension of the subject is received, wherein the step of quantitatively processing the attribute is based on the received data indicating at least one of the level of knee extension or quadriceps contraction task of the subject from a sitting position or supine position. In an example, the step of deriving an attribute from the sensor data comprises extracting features as shown in FIG. 6. The step of quantitatively processing the attribute involves an exercise program. The frailty score that is obtained may then be sent wirelessly. In an example, it includes remotely assisting the exercise program of the subject.

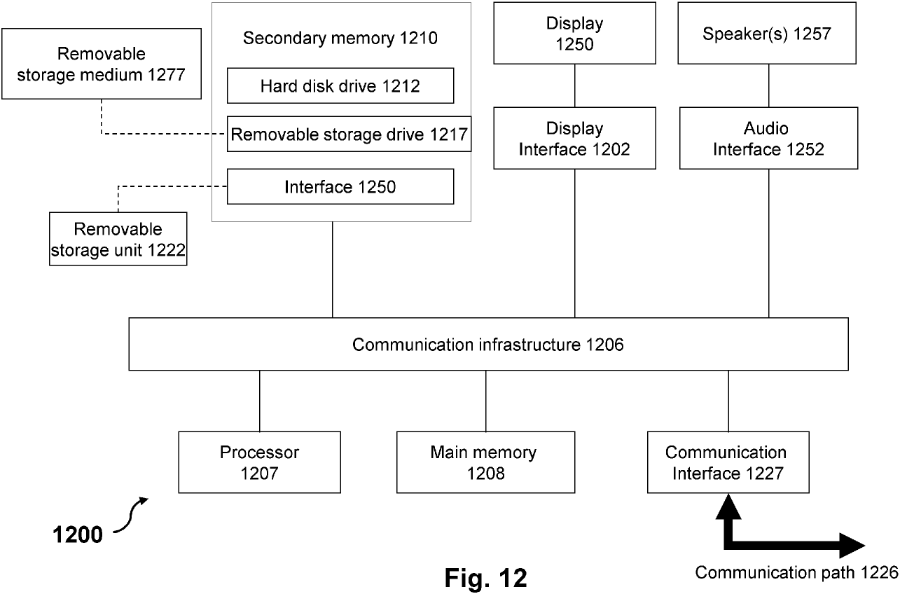

FIG. 12 depicts an exemplary computing device 1200, hereinafter interchangeably referred to as a computer system 1200, where one or more such computing devices 1200 may be used to execute the method of FIG. 5. The exemplary computing device 1200 can be used to implement the system 100 shown in FIG. 1. The following description of the computing device 1200 is provided by way of example only and is not intended to be limiting.

As shown in FIG. 12, the example computing device 1200 includes a processor 607 for executing software routines. Although a single processor is shown for the sake of clarity, the computing device 1200 may also include a multi-processor system. The processor 1207 is connected to a communication infrastructure 1206 for communication with other components of the computing device 1200. The communication infrastructure 1206 may include, for example, a communications bus, cross-bar, or network.

The computing device 1200 further includes a main memory 1208, such as a random access memory (RAM), and a secondary memory 1210. The secondary memory 610 may include, for example, a storage drive 1212, which may be a hard disk drive, a solid state drive or a hybrid drive and/or a removable storage drive 1217, which may include a magnetic tape drive, an optical disk drive, a solid state storage drive (such as a USB flash drive, a flash memory device, a solid state drive or a memory card), or the like. The removable storage drive 1217 reads from and/or writes to a removable storage medium 1277 in a well-known manner. The removable storage medium 1277 may include magnetic tape, optical disk, non-volatile memory storage medium, or the like, which is read by and written to by removable storage drive 1217. As will be appreciated by persons skilled in the relevant art(s), the removable storage medium 1277 includes a computer readable storage medium having stored therein computer executable program code instructions and/or data.

In an alternative implementation, the secondary memory 1210 may additionally or alternatively include other similar means for allowing computer programs or other instructions to be loaded into the computing device 1200. Such means can include, for example, a removable storage unit 1222 and an interface 1250. Examples of a removable storage unit 1222 and interface 1250 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 1222 and interfaces 1250 which allow software and data to be transferred from the removable storage unit 1222 to the computer system 1200.

The computing device 1200 also includes at least one communication interface 627. The communication interface 1227 allows software and data to be transferred between computing device 1200 and external devices via a communication path 1227. In various embodiments of the inventions, the communication interface 1227 permits data to be transferred between the computing device 1200 and a data communication network, such as a public data or private data communication network. The communication interface 1227 may be used to exchange data between different computing devices 600 which such computing devices 1200 form part an interconnected computer network. Examples of a communication interface 1227 can include a modem, a network interface (such as an Ethernet card), a communication port (such as a serial, parallel, printer, GPIB, IEEE 1394, RJ45, USB), an antenna with associated circuitry and the like. The communication interface 1227 may be wired or may be wireless. Software and data transferred via the communication interface 1227 are in the form of signals which can be electronic, electromagnetic, optical or other signals capable of being received by communication interface 1227. These signals are provided to the communication interface via the communication path 1227.

As shown in FIG. 12, the computing device 1200 further includes a display interface 1202 which performs operations for rendering images to an associated display 650 and an audio interface 1252 for performing operations for playing audio content via associated speaker(s) 1257.

As used herein, the term “computer program product” may refer, in part, to removable storage medium 1277, removable storage unit 1222, a hard disk installed in storage drive 1212, or a carrier wave carrying software over communication path 1227 (wireless link or cable) to communication interface 1227. Computer readable storage media refers to any non-transitory, non-volatile tangible storage medium that provides recorded instructions and/or data to the computing device 1200 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 1200. Examples of transitory or non-tangible computer readable transmission media that may also participate in the provision of software, application programs, instructions and/or data to the computing device 1200 include radio or infra-red transmission channels as well as a network connection to another computer or networked device, and the Internet or Intranets including e-mail transmissions and information recorded on Websites and the like.

The computer programs (also called computer program code) are stored in main memory 608 and/or secondary memory 1210. Computer programs can also be received via the communication interface 1227. Such computer programs, when executed, enable the computing device 1200 to perform one or more features of embodiments discussed herein. In various embodiments, the computer programs, when executed, enable the processor 1207 to perform features of the above-described embodiments. Accordingly, such computer programs represent controllers of the computer system 1200.

Software may be stored in a computer program product and loaded into the computing device 1200 using the removable storage drive 1217, the storage drive 1212, or the interface 1250. The computer program product may be a non-transitory computer readable medium. Alternatively, the computer program product may be downloaded to the computer system 1200 over the communications path 627. The software, when executed by the processor 1207, causes the computing device 600 to perform the necessary operations to execute the method 500 as shown in FIG. 5.

It is to be understood that the embodiment of FIG. 12 is presented merely by way of example to explain the operation and structure of the system 100. Therefore, in some embodiments one or more features of the computing device 1200 may be omitted. Also, in some embodiments, one or more features of the computing device 1200 may be combined together. Additionally, in some embodiments, one or more features of the computing device 1200 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

The program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

This application is based upon and claims the benefit of priority from Singapore patent application No. 10202005961P filed on 22 June, 2020, the disclosure of which is incorporated herein in its entirety by reference.

What is claimed is:

1. A method of determining a frailty score of a subject, the method comprising:
deriving an attribute from sensor data including at least one of EMG data or motion date of the subject, the sensor data being obtained from at least one limb of the subject;
deriving information indicating that how the subject is responding to the rehabilitation or exercise program based on the attribute; and
determining the frailty score of the subject based on the information indicating that how the subject is responding to the rehabilitation or exercise program.

2. The method according to claim 1, further comprising:
obtaining the sensor data from at least a quadriceps of the subject, and
wherein the frailty score indicates an objective quadriceps muscle strength.

3. The method according to claim 1 or 2, wherein the information indicating that how the subject is responding to the rehabilitation or exercise program includes at least one of a range of motion of the subject or velocity of movement of the subject, muscle strength, similarity score of an injured limb and an uninjured limb.

4. The method according to claim 1, further comprising:
receiving time taken for the subject to get up from a sitting position,
wherein the information indicating that how the subject is responding to the rehabilitation or exercise program is derived based on the time taken for the subject to get up from the sitting position.

5. The method according to claim 1, further comprising:
receiving data indicating an amount of muscle contraction of the subject during getting up from a sitting position,
wherein the information indicating that how the subject is responding to the rehabilitation or exercise program is derived based on repeated movement to get up from the sitting position, or predetermined period of time or a predetermined number of repetition to get up from sitting position.

6. The method according to claim 1, further comprising: receiving time taken for the subject to perform at least one of quadriceps contraction task and knee extension from a sitting position or supine position.

7. The method according to claim 1, further comprising:
receiving data indicating the amount of muscle contraction and level of knee extension or quadriceps contraction task of the subject,
wherein the information indicating that how the subject is responding to the rehabilitation or exercise program is derived based on the data indicating the amount of muscle contraction and the level of knee extension or quadriceps contraction task during a predetermined period of time or a predetermined number of repetition.

8. The method according to claim 1, wherein the step of deriving an attribute from the sensor data comprises extracting features.

9. The method according to claim 1, wherein the information indicating that how the subject is responding to the rehabilitation or exercise program includes an exercise program.

10. The method according to claim 1, further comprising: remotely assisting the exercise program of the subject.

11. The method according to claim 1, further comprising:
sending the frailty score wirelessly.

12. An apparatus of determining a frailty score of a subject, the apparatus comprising:
at least one processor; and
at least one memory including computer program code;
the at least one memory and the computer program code configured to, with at least one processor, cause the apparatus at least to:
derive an attribute from sensor data including at least one of EMG data or motion date of the subject, the sensor data being obtained from at least one limb of the subject;
derive information indicating that how the subject is responding to the rehabilitation or exercise program based on the attribute; and
determine the frailty score of the subject based on the information indicating that how the subject is responding to the rehabilitation or exercise program.

13. The apparatus according to claim 12, wherein the at least one memory and the computer program code configured to, with at least one processor, cause the apparatus at least to:
obtain the sensor data from at least a quadriceps of the subject, and
wherein the frailty score indicates an objective quadriceps muscle strength.

14. The apparatus according to claim 12 or 13, wherein the at least one memory and the computer program code configured to, with at least one processor, cause the apparatus at least to:
at least one of a range of motion of the subject or velocity of movement of the subject, muscle strength, similarity score of an injured limb and an uninjured limb.

15. The apparatus according to claim 12, wherein the at least one memory and the computer program code configured to, with at least one processor, cause the apparatus at least to:
receive time taken for the subject to get up from a sitting position,
wherein the apparatus is derived based on the time taken for the subject to get up from the sitting position.

16. The apparatus according to claim 12, wherein the at least one memory and the computer program code configured to, with at least one processor, cause the apparatus at least to:
receive data indicating a level of knee extension or quadriceps contraction task of the subject,
wherein the apparatus is derived based on the received data indicating the level of knee extension or quadriceps contraction task.

17. The apparatus according to claim 12, wherein the at least one memory and the computer program code configured to, with at least one processor, cause the apparatus at least to:
extract features to derive an attribute from the sensor data.

18. The apparatus according to claim 12, wherein the at least one memory and the computer program code configured to, with at least one processor, cause the apparatus at least to:
quantitatively process the attribute that involves an exercise program.

19. The apparatus according to claim 12, wherein the at least one memory and the computer program code configured to, with at least one processor, cause the apparatus at least to:
send the frailty score wirelessly.

20. The apparatus according to claim 12, wherein the at least one memory and the computer program code configured to remotely assist the exercise program of the subject.
